(19) United States
(12) Patent Application Publication
(10) Pub. No.: US 2011/0263889 A1
(43) Pub. Date: Oct. 27, 2011

(54) PROCESSES FOR THE PREPARATION OF ESTERS

(75) Inventors: Martin Charles Bowden, Berkshire (GB); David Anthony Jackson, Munchwilen (CH); Alexandre Christian Saint-Dizier, Monthey (CH)

(73) Assignee: SYNGENTA CROP PROTECTION, INC., Greensboro, NC (US)

(21) Appl. No.: 12/992,833

(22) PCT Filed: May 11, 2009
(86) PCT No.: PCT/EP09/55649

§ 371 Date: Feb. 3, 2011

(30) Foreign Application Priority Data
May 14, 2008 (GB) 0808767.8

(51) Int. Cl. C07C 67/08

(52) U.S. Cl. 560/124

(57) ABSTRACT

The invention relates to a process for the preparation of synthetic pyrethroid insecticides. The process involves coupling of carboxylic acid (IV) with an alcohol (V) in the presence of a catalyst wherein R1, R2, R3 and Ar are defined in the specification.

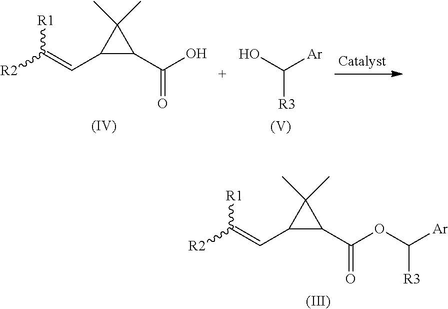

The present invention relates to a process for the preparation of certain pyrethroid compounds useful as insecticides, as well as a process for the preparation of intermediates useful in the synthesis of such compounds.

Synthetic pyrethroid insecticides of the formula (I) are disclosed in GB2000764

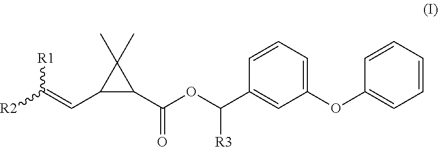

wherein
one of R1 and R2 represents a group of formula: W˜(CF₂)m˜ where W represents an atom of hydrogen, fluorine or chlorine and m has the value one or two, and the other of R1 and R2 represents an atom of fluorine, chlorine or bromine, and R3 represents an atom of hydrogen or the cyano or ethynyl group.

Synthetic pyrethroid insecticides of the formula (II) are disclosed in U.S. Pat. No. 4,405,640

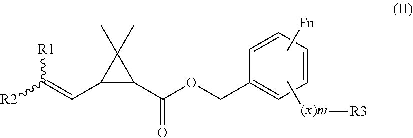

wherein
R1 and R2 are each selected from methyl, halomethyl, and halo; X is oxygen, sulphur, sulphonyl or a group NR4 where R4 represents hydrogen, lower alkyl or lower carboxylic acyl; R3 is lower alkyl, lower alkenyl or benzyl; m has the value zero to one, and n has a value from one to four.

The present invention seeks to provide processes for the preparation of these and other pyrethroids.

It has now surprisingly been found that these compounds may advantageously obtained by coupling the corresponding alcohol and carboxylic acid in the presence of a catalyst, as described below.

The present invention provides a process for the preparation of compounds of formula (III)

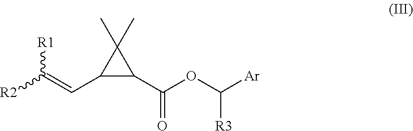

wherein
R1 is hydrogen, alkyl, haloalkyl, or halogen;
R2 is hydrogen, alkyl, haloalkyl, or halogen;
R3 is hydrogen, halogen, hydroxyl, nitro, cyano, optionally substituted alkyl, optionally substituted alkenyl, or optionally substituted alkynyl;
Ar is an optionally substituted aryl group
comprising reacting a carboxylic acid of formula (IV)

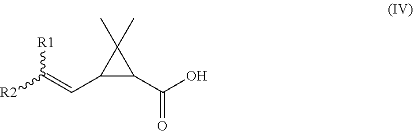

with an alcohol of the formula (V)

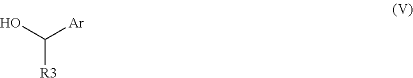

in the presence of a catalyst selected from zirconium containing catalysts, hafnium containing catalysts, iron containing catalysts, cobalt containing catalysts, tin containing catalysts, titanium containing catalysts, ammonium salt catalysts and boronic acid containing catalysts.

Preferably, R1 and R2 are independently selected from hydrogen, halogen, C1-C4 alkyl and C1-C4 haloalkyl.

Preferably, R3 is selected from hydrogen, halogen, hydroxyl, nitro, cyano, C1-C4 alkyl and C1-C4 haloalkyl, more preferably hydrogen, cyano or C3-C4 alkynyl.

Preferably Ar is phenyl substituted with one or more groups selected from hydrogen, halogen, hydroxyl, nitro, cyano, C1-C4 alkyl, C1-C4 haloalkyl, and phenoxy.

Halo is fluoro, chloro or bromo.

Each alkyl moiety is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. Preferred alkyl groups have from 1 to 6 carbon atoms.

Haloalkyl refers to an alkyl moiety as defined above wherein at least one hydrogen atom is substituted for a halo atom.

Aryl refers to a phenyl or napthyl group.

Heteroaryl refers to a mono- or bicyclic ring system wherein each ring comprises from 5 to 7 ring member atoms, and from 1 to 3 heteroatoms independently selected from O, N and S. Examples of heteroaryl groups are pyridine, pyrrole, furan, pyrazole, imidazole and oxazole.

Alkenyl refers to a straight or branched group consisting of carbon and hydrogen atoms comprising at least one carbon-carbon double bond. Examples are ethenyl, prop-1-enyl, prop-2-enyl, but-1-enyl, but-2-enyl and but-3-enyl. Both cis- and trans-groups are contemplated. Preferred alkenyl groups have from 2 to 6 carbon atoms.

Alkynyl refers to a straight or branched group consisting of carbon and hydrogen atoms comprising at least one carbon-carbon triple bond. Examples are ethynyl, prop-1-ynyl, prop-2-ynyl, but-1-ynyl, but-2-ynyl and but-3-ynyl. Preferred alkynyl groups have from 2 to 6 carbon atoms.

Cycloalkyl includes cyclopropyl, cyclobutyl, cyclopentyl and cyclohexyl.

When present, each optional substituent on aryl or on heteroaryl is, independently, preferably selected from alkyl, alkenyl, alkynyl, alkoxyl, alkenyloxy, aryloxy, thioalkyl, amino, alkylamino, dialkylamino, aralkyl, acetamido, n-alkyl acetamido, alkylsulfonyl, halo, hydroxyl, cyano and nitro. From one to the maximum possible number of substituents may be present.

Preferably, one of R1 and R2 is halo, and the other is haloalkyl. More preferably, one of R1 and R2 is chloro, and the other is haloalkyl, e.g. halo-C1-C4-alkyl. More preferably, one of R1 and R2 is halo, and the other is trifluoromethyl. Most preferably, one of R1 and R2 is chloro, and the other is trifluoromethyl.

Preferably, the substituents on the cyclopropane ring are in the cis-stereochemistry. That is, the compound of formula (III) has the stereochemistry (IIIa)

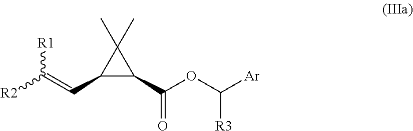

Preferably, the compound of formula (III) has the formula (IIIb)

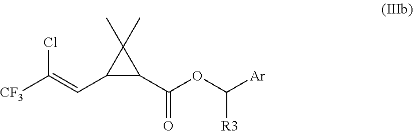

Very preferably, the compound of formula (III) has the formula (IIIc)

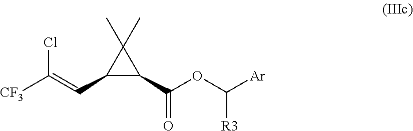

In a preferred embodiment, Ar is an optionally substituted phenyl group.

In a highly preferred embodiment, Ar is a phenoxyphenyl group. More preferably, Ar is a 3-phenoxyphenyl group. In this embodiment, it is preferred that R3 is cyano or ethynyl. It is more preferred that R3 is cyano.

For example,

One of R1 and R2 is halo, and the other is halo-C1-C4-alkyl;
R3 is cyano or ethynyl;
Ar is a phenoxyphenyl group.

Very highly preferably, the compound of formula (III) has the formula (IIId)

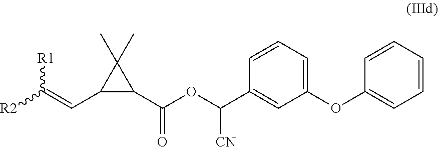

In an alternative preferred embodiment, Ar is a group of the formula (VI)

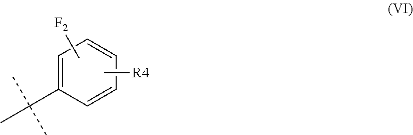

wherein R4 is selected from alkyl, alkenyl, alkynyl, alkoxyl, alkenyloxy, aryloxy, thioalkyl, amino, alkylamino, dialkylamino, aralkyl, acetamido, n-alkyl acetamido, alkylsulfonyl, halo, hydroxyl, cyano and nitro. Preferably, R4 is alkyl of 1 to 4 carbon atoms, alkenyl of three to five carbon atoms, methoxy, ethoxy, allyloxy, ethylthio, ethanesulphonyl, benzyl, dimethylamino, ethylamino, acetamido or n-methylacetamido. Very preferably, R4 is methyl. Preferably, R4 is in the 4-position.

In this embodiment, it is preferred that R3 is hydrogen.

For example,

One of R1 and R2 is halo, and the other is halo-C1-C4-alkyl;
R3 is hydrogen;
Ar is a group of the formula (VI)

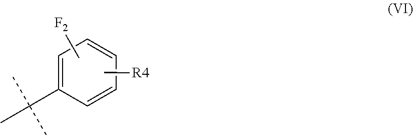

R4 is alkyl of 1 to 4 carbon atoms, alkenyl of three to five carbon atoms, methoxy, ethoxy, allyloxy, ethylthio, ethanesulphonyl, benzyl, dimethylamino, ethylamino, acetamido or n-methylacetamido.

Very highly preferably, the compound of formula (III) has the formula (IIIe)

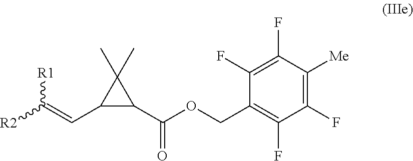

According to a very highly preferred embodiment, the invention provides a process for the preparation of tefluthrin (VII)

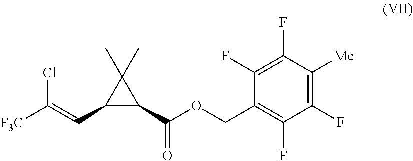

comprising reacting a carboxylic acid of formula (VIII) with an alcohol of formula (IX)

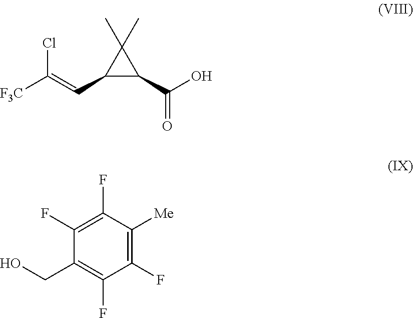

in the presence of a catalyst selected from zirconium containing catalysts, hafnium containing catalysts, iron containing catalysts, cobalt containing catalysts, tin containing catalysts, titanium containing catalysts, ammonium salt catalysts and boronic acid containing catalysts.

According to an alternative very highly preferred embodiment, the invention provides a process for the preparation of lambda cyhalothrin (X)

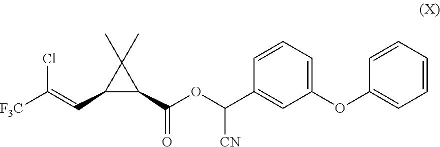

comprising reacting a carboxylic acid of formula (VIII) with an alcohol of formula (XI)

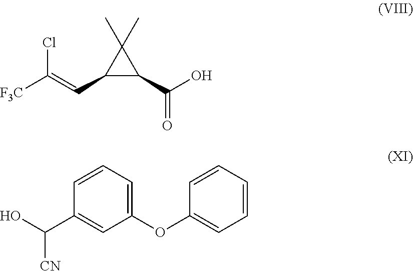

in the presence of a catalyst selected from zirconium containing catalysts, hafnium containing catalysts, iron containing catalysts, cobalt containing catalysts, tin containing catalysts, titanium containing catalysts, ammonium salt catalysts and boronic acid containing catalysts.

Compound (XI) may be generated in situ.

The present invention also relates to a process substantially as described herein with reference to the examples.

The present invention also relates to a compound obtainable by the process described herein. The present invention also relates to a compound obtained by the processes described herein.

### Enantiomers

The processes of the invention may be used to prepare enantiomerically enriched or pure forms of the compounds. In a preferred embodiment, the invention provides a process for the preparation of gamma-cyhalothrin (S)-a-cyano-3-phenoxybenzyl(Z)-(1R,3R)-3-(2-chloro-3,3,3-trifluoro-1-propenyl)-2,2-dimethylcyclopropane carboxylate) (XIV)

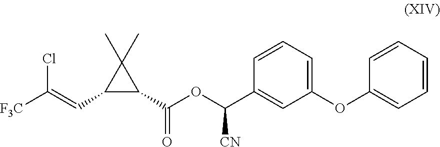

### Solvent

The reaction of the invention is optionally (and preferably) conducted in a suitable solvent. Suitable solvents include, but are not limited to, linear, branched or cyclic aliphatic hydrocarbons, such as ligroin or cyclohexane, pentane, hexane, heptane, octane, as well as aromatic solvents, such as benzene, toluene, xylene, monochlorobenzene, dichlorobenzene, trichlorobenzene.

A preferred solvent is xylene.

### Temperature

The reaction of the invention may be carried out at a temperature such that an acceptable rate of reaction is attained. Preferably, the reaction is conducted at a temperature of from 0° C. to 200° C. More preferably, the reaction is conducted at a temperature of from 50° C. to 180° C. More preferably, the reaction is conducted at a temperature of from 100° C. to 170° C. More preferably, the reaction is conducted at a temperature of from 130° C. to 150° C.

### Removal of Water

Preferably, provision is made for removal of water from the reaction mixture, e.g. removal of water prior to completion of the reaction. Water may be removed from the reaction continuously. A suitable method is azeotropic removal of water. Suitable apparatus for conducting azeotropic removal of water will be known to those skilled in the art. We have found that removal of water is highly desirable in order to achieve a commercially useful conversion to product.

### Catalyst

Preferred classes of catalyst are i) zirconium containing catalysts, ii) hafnium containing catalysts, iii) iron containing catalysts, iv) cobalt containing catalysts, v) tin containing catalysts, vi) titanium containing catalysts, vii) ammonium salt catalysts and boronic acid containing catalysts.

Preferably the catalyst is a zirconium containing catalyst. Preferred zirconium containing catalysts are zirconium (IV) compounds. More preferred are zirconium (IV) halides, zirconium (IV) oxyhalides, zirconium (IV) alkoxides, zirconocene dichloride, and the solvent complexes of these species, especially the tetrahydrofuran complexes. Very preferred are ZrCl₄, ZrOCl₂, Zr(OⁱPr₄), Zirconocene dichloride, ZrBr₄, and ZrCl₄.(THF)₂.

The most preferred catalyst is ZrCl₄.

Preferred hafnium containing catalysts are hafnium (IV) compounds. More preferred are hafnium (IV) halides and the solvent complexes thereof. More preferred are HfCl₄ and HfCl₄.(THF)₂.

Preferred iron containing catalysts are iron (III) compounds. More preferred are iron (III) halides. More preferred is FeCl₃.

A preferred cobalt-containing catalyst is K₅CoW₁₂O₄₀.

Preferred tin containing catalysts are tin (IV) compounds. More preferred are tin (IV) halides. More preferred is SnCl₄.

Preferred titanium compounds are titanium (IV) compounds. More preferred are titanium (IV) halides and titanium (IV) alkoxides. More preferred are Ti(OⁱPr)₄ and TiCl₄.

A preferred ammonium salt catalyst is Ph₂NH₂OTf.

Examples of the boronic acids for boronic acid containing catalysts include boric acid, phenylboronic acid, 2-methylphenylboronic acid, 3-methylphenylboronic acid, 4-methylphenylboronic acid, 2,3-dimethylphenylboronic acid, 4-dimethylphenylboronic acid, 2,5-dimethylphenylboronic acid, 2-ethylphenylboronic acid, 4-n-propylphenylboronic acid, 4-isopropylphenylboronic acid, 4-n-butylphenylboronic acid, 4-tert-butylphenylboronic acid, 1-naphthylboronic acid, 2-naphthylboronic acid, 2-biphenylboronic acid, 3-biphenylboronic acid, 4-biphenylboronic acid, 2-fluoro-4-biphenylboronic acid, 2-fluorenylboronic acid, 9-fluorenylboronic acid, 9-phenanthrenylboronic acid, 9-anthracenylboronic acid, 1-pyrenylboronic acid, 2-trifluoromethylphenylboronic acid, 3-trifluoromethylphenylboronic acid, 4-trifluorophenylboronic acid, 3,5-bis(trifluoromethyl)phenylboronic acid, 2-methoxyphenylboronic acid, 3-methoxyphenylboronic acid, 4-methoxyphenylboronic acid, 2,5-dimethoxyphenylboronic acid, 4,5-dimethoxyphenylboronic acid, 2,4-dimethoxyphenylboronic acid, 2-ethoxyphenylboronic acid, 3-ethoxyphenylboronic acid, 4-ethoxyphenylboronic acid, 4-phenoxyboronic acid, 4-methylenedioxyphenylboronic acid, 2-fluorophenylboronic acid, 3-fluorophenylboronic acid, 4-fluorophenylboronic acid, 2,4-difluorophenylboronic acid, 2,5-difluorophenylboronic acid, 4,5-difluorophenylboronic acid, 3,5-difluorophenylboronic acid, 2-formylphenylboronic acid, 3-formylphenylboronic acid, 4-formylphenylboronic acid, 3-formyl-4-methoxyphenylboronic acid, 2-cyanophenylboronic acid, 3-cyanophenylboronic acid, 4-cyanophenylboronic acid, 3-nitrophenylboronic acid, 3-acetylphenylboronic acid, 4-acetylphenylboronic acid, 3-trifluoroacetylphenylboronic acid, 4-trifluoroacetylphenylboronic acid, 4-methylthiophenylboronic acid, 4-vinylphenylboronic acid, 3-carboxyphenylboronic acid, 4-carboxyphenylboronic acid, 3-aminophenylboronic acid, 2-(N,N-dimethylamino)phenylboronic acid, 3-(N,N-dimethylamino)phenylboronic acid, 4-(N,N-dimethylamino)phenylboronic acid, 2-(N,N-diethylamino)phenylboronic acid, 3-(N,N-diethylamino)phenylboronic acid, 4-(N,N-diethylamino)phenylboronic acid, 2-(N,N-dimethylaminomethyl)phenylboronic acid, furan-2-boronic acid, furan-3-boronic acid, 4-formyl-2-furanboronic acid, dibenzofuran-4-boronic acid, benzofuran-2-boronic acid, thiophene-2-boronic acid, thiophene-3-boronic acid, 5-methylthiophene-2-boronic acid, 5-chlorothiophene-2-boronic acid, 4-methylthiophene-2-boronic acid, 5-methylthiophene-2-boronic acid, 2-acetylthiophene-5-boronic acid, 5-methylthiophene-2-boronic acid, benzothiophene-2-boronic acid, dibenzothiophene-4-boronic acid, pyridine-3-boronic acid, pyridine-4-boronic acid, pyrimidine-5-boronic acid, quinoline-8-boronic acid, isoquinoline-4-boronic acid, 4-benzenebis(boronic acid), phenylboronic acid-pinacol ester, and 4-cyanophenylboronic acid-pinacol ester.

A preferred class of boronic acids are aryl boronic acids. e.g. 2-(N,N-dimethylaminomethyl)phenylboronic acid and 3,5-trifluoromethylphenylboronic acid. An alternative preferred boronic acid is boric acid.

Preferably the catalyst is a zirconium containing catalyst, a hafnium containing catalyst, an ammonium salt catalyst or a boronic acid containing catalyst, particularly a zirconium containing catalyst or an ammonium salt catalyst. More preferably the catalyst is a zirconium containing catalyst.

Preferably, the catalyst comprises or consists of ZrCl₄ or a solvent complex thereof, HfCl₄ or a solvent complex thereof, Ph₂NH₂⁺OTf⁻ or boric acid. More preferably the catalyst comprises or consists of ZrCl₄ or a solvent complex thereof or Ph₂NH₂⁺OTf⁻.

Usually one type of catalyst will be used in a reaction. However, the invention also covers reactions in which more than one type of catalyst is used, e.g. either separately, sequentially or simultaneously. For example, more than one type of zirconium catalyst may be used or a zirconium catalyst may be used in combination with a boronic acid catalyst.

### Amount of Catalyst

Preferably, the amount of catalyst employed is up to 50 mol % based on the amount of carboxylic acid (IV). More preferably, the amount of catalyst employed is up to 25 mol % based on the amount of carboxylic acid (IV). More preferably, the amount of catalyst employed is up to 15 mol % based on the amount of carboxylic acid (IV).

Preferably, the amount of catalyst employed is at least 0.01 mol % based on the amount of carboxylic acid (IV). More preferably, the amount of catalyst employed is at least 0.1 mol % based on the amount of carboxylic acid (IV). More preferably, the amount of catalyst employed is at least 1 mol % based on the amount of carboxylic acid (IV). More preferably, the amount of catalyst employed is at least 5 mol % based on the amount of carboxylic acid (IV)

Preferably, the amount of catalyst employed is between 0.01 and 50 mol % based on the amount of carboxylic acid (IV). More preferably, the amount of catalyst employed is between 0.1 and 25 mol % based on the amount of carboxylic acid (IV). More preferably, the amount of catalyst employed is between 5 and 15 mol % based on the amount of carboxylic acid (IV).

Preferably, the amount of catalyst employed is up to 50 mol % based on the amount of alcohol (V). More preferably, the amount of catalyst employed is up to 25 mol % based on the amount of alcohol (V). More preferably, the amount of catalyst employed is up to 15 mol % based on the amount of alcohol (V).

Preferably, the amount of catalyst employed is at least 0.01 mol % based on the amount of alcohol (V). More preferably, the amount of catalyst employed is at least 0.1 mol % based on the amount of alcohol (V). More preferably, the amount of catalyst employed is at least 1 mol % based on the amount of alcohol (V). More preferably, the amount of catalyst employed is at least 5 mol % based on the amount of alcohol (V).

Preferably, the amount of catalyst employed is between 0.01 and 50 mol % based on the amount of alcohol (V). More preferably, the amount of catalyst employed is between 0.1 and 25 mol % based on the amount of alcohol (V). More preferably, the amount of catalyst employed is between 5 and 15 mol % based on the amount of alcohol (V).

### Synthesis of Starting Materials

Suitable methods for the preparation of carboxylic acids (IV) and alcohols (V) are disclosed in U.S. Pat. No. 4,405,640 and GB2000764. Other methods will be apparent to those skilled in the art.

### Workup and Isolation of Products

Workup of the reaction mixture is achieved according to well known procedures of synthetic organic chemistry. For example, an aqueous workup may be achieved by the addition of water (or other aqueous solution), and extraction of the desired product with a suitable organic solvent.

Alternatively, the product may be isolated by removing any solvent present by distillation, e.g. under reduced pressure.

Purification of the product may be achieved by any one of a number of methods, e.g. distillation, recrystallization and chromatography.

The present invention will now be described by way of the following non-limiting examples. Those skilled in the art will promptly recognize appropriate variations from the procedures both as to reactants and as to reaction conditions and techniques.

All references mentioned herein are incorporated by reference in their entirety. All aspects and preferred features of the invention may be combined with each other, except where this is evidently not possible.

### EXAMPLES

### Example 1

### Reaction Sequence

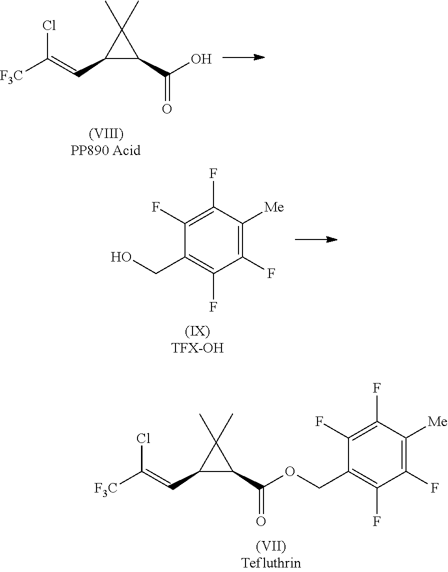

| Solvent | Conditions | Time | Catalyst | Conversion |
|---|---|---|---|---|
| Xylene | Reflux, Dean & Stark | 3 hours | ZrCl₄ | 2% |
| | | | (1 Mol %) | |
| Xylene | Reflux, Dean & Stark | 3 hours | ZrCl₄ | 100% |
| | | | (10 Mol %) | |

Reaction of PP890 acid (VIII) and TFX-OH (IX) for 3 hours at 140° C. in the presence of a small amount of zirconium tetrachloride (1 mol %) gave very little conversion (2%). Addition of more catalyst (10 mol %) resulted in clean and rapid conversion to the desired tefluthrin product (VII).

The identity of the product was confirmed by GCMS and NMR comparison with authentic material.

Further experimental details:

| | Actual | Strength | 100% | Mol | | Mol. |
|---|---|---|---|---|---|---|
| Materials | Wt (g) | (%) | Wt. (g) | Wt. | mmol. | Ratio |
| TFXOH | 8.0 | 96.6 | 7.8 | 194 | 40 | 1.0 |
| PP890 | 9.8 | 99.5 | 9.7 | 243 | 40 | 1.0 |
| ZrCl₄ | 0.1 | 98 | 0.1 | 233 | 4 | 0.1 |
| Xylene | 100 ml | — | — | — | — | — |

### Procedure

A 250 ml three neck round bottom flask was fitted with a magnetic flea, thermometer, oil bath, condenser, and Dean & Stark apparatus filled with 3A molecular sieves 8-12 mesh (with 10 ml xylene). The system was purged with nitrogen and vented to atmosphere.

TFXOH (8.0 g), PP890 (9.8 g), xylene (100 ml) and ZrCl₄ (0.01 g) were charged to the flask. The mixture was heated to reflux (145° C.) and held on temperature for 5 hrs. Further ZrCl₄ catalyst (0.09 g) was added, the liquid in the Dean & Stark receiver replaced with fresh xylene, and the mixture refluxed for a further 4.5 hr.

Reaction was monitored via GC:

Conversion after 1 hr (0.1 equivalent catalyst)=65%
Conversion after 3 hr (0.1 equivalent catalyst)=90%
Conversion after 4.5 hr (0.1 equivalent catalyst)=99%

A portion (20 ml) of the reaction mass was washed with water (2×10 ml), dried (MgSO₄) and concentrated in vacuo. The identity of the product was confirmed by GC, GCMS and NMR.

### Example 2

### Reaction Sequence

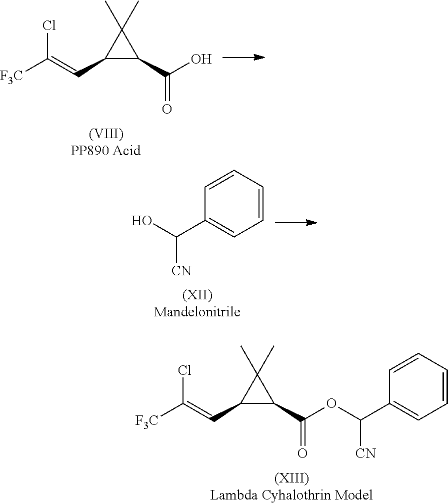

The procedure of Example 1 was repeated varying the substrate employed.

| Solvent | Conditions | Time | Catalyst | Conversion |
|---|---|---|---|---|
| Xylene | Reflux, Dean & Stark | 3 hours | None | 2% |
| Xylene | Reflux, Dean & Stark | 3 hours | ZrCl₄ | 20% |
| | | | (10 Mol %) | |

The cyanohydrin needed for cyhalothrin is not readily available, and is generated in situ. Madelonitrile is readily available and structurally similar, so this was used as a model for the cyhalothrin system.

Little reaction occurred in the absence of any catalyst (2% conversion after 3 hours at 140° C.

Significant conversion was achieved in the presence of zirconium tetrachloride catalyst (10 mol %).

### Example 3

### Reaction Sequence

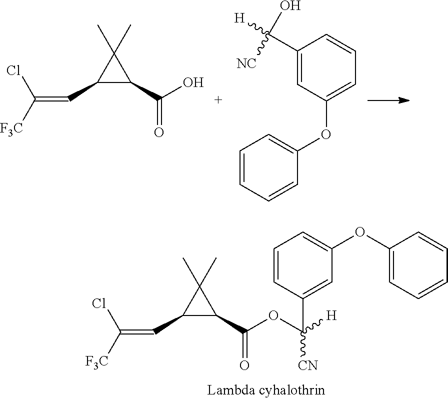

| | Actual | Strength | 100% | Mol | | Mol. |
|---|---|---|---|---|---|---|
| Materials | Wt (g) | (%) | Wt. (g) | Wt. | mmol. | Ratio |
| 3-phenoxy | 0.66 | 70 | 0.462 | 225 | 2.05 | 1.0 |
| benzylaldehyde | | | | | | |
| cyandohydrin | | | | | | |
| PP890 | 0.5 | 100 | 0.498 | 242.5 | 2.05 | 1.0 |
| Diphenylammonium | 95 | 0.063 | 0.063 | 309 | 0.205 | 0.1 |
| triflilate | | | | | | |
| Xylene | 20 ml | | | | | 62.4 |

A 50 ml three neck round bottom flask was fitted with a magnetic flea, thermometer, oil bath, condenser, and Dean & Stark apparatus filled with 3A molecular sieves 8-12 mesh (with 10 ml xylene). The system was purged with nitrogen and vented to atmosphere.

3-phenoxy benzylaldehyde cyandohydrin (0.66 g), PP890 (0.5 g), xylene (20 ml) and Diphenylammonium triflilate catalyst (67 mg) were charged to the flask. The mixture was heated to reflux (˜143 deg C.) and held on temperature for 10 hrs. The reaction was monitored via GC.

Conversion=40% (of two cyhalothrin isomers) after 10 hr.

GC/MS: The product had same retention time, molecular ion (M⁺ 449) and fragmentation pattern as found with authentic material.

NMR: The NMR data was consistent with that for authentic material.

### Example 4

### Reaction Sequence

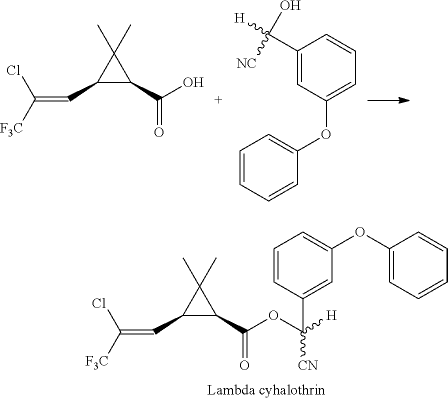

Direct acylation of 3-phenoxy benzylaldehyde cyanohydrin with PP890 was carried out using conditions described in Example 3. A selection of catalysts was screened:

| Catalyst | Solvent | Conditions | Results |
|---|---|---|---|
| None | Xylene | Vigorous reflux | No reaction. |
| | | with Dean & | |
| | | Stark set-up | |
| | | 5.5 hr | |
| B(OH)₃ | Xylene | Vigorous reflux | 20% conversion |
| | | with Dean & | 5% in total of the two |
| | | Stark set-up | cyhalothrin isomers and 15% of |
| | | 5.5 hr | two unidentified impurities |
| HfCl₄•2THF | Xylene | Vigorous reflux | 20% conversion |
| | | with Dean & | 10% each of two cyhalothrin |
| | | Stark set-up | isomers |
| | | 5.5 hr | |
| Ph₂NH₂⁺OTf | Xylene | Vigorous reflux | 46% conversion |
| | | with Dean & | Total of 37% of the two |
| | | Stark set-up | cyhalothrin isomers and 9% of |
| | | overnight | the two unidentified impurities |

cyhalothrin was successfully formed in the presence of catalyst, and no reaction was observed without catalysis. All three catalysts used gave some conversion to the desired product, but diphenylammonium trifilate was clearly the most effective.

Racemic cyanohydrin and PP890 were used, so the product was formed as a 1:1 mixture of diasteromers:

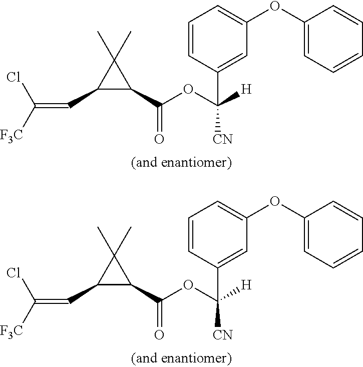

The product was isolated and characterised by NMR and GC/MS. Spectral data for the material were consistent with the desired product.

Two long retention time impurities were seen by GCMS when boric acid and diphenylammonium trifilate were used as catalysts. The two impurities had the same molecular weight suggesting they were isomeric with each other. Their molecular weight (MW 467) was 18 higher than that of the desired product (MW 449). It seems likely therefore that they are hydrated derivatives of the target molecules. E.g. product analogue where the nitrile group has been hydrolysed to an amide:

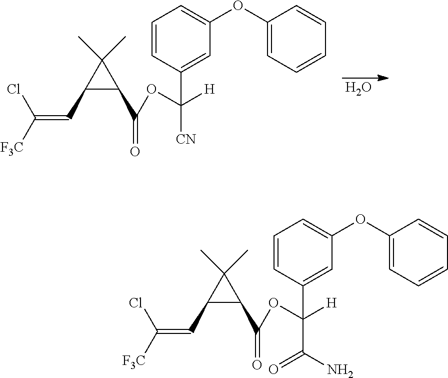

The different behaviour observed between catalysts is thought to be due to differences in the nitrogen purging used. Less efficient purging was used with the boric acid and triflate experiments, which may have allowed some ingress of water (resulting in product hydrolysis).

1. A process for the preparation of compounds of formula (III) 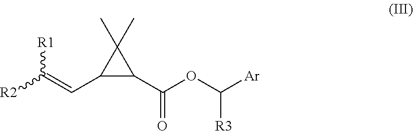
wherein
R1 is hydrogen, alkyl, haloalkyl, or halogen;
R2 is hydrogen, alkyl, haloalkyl, or halogen;
R3 is hydrogen, cyano or alkynyl;
Ar is an optionally substituted aryl or heteroaryl group
comprising reacting a carboxylic acid of formula (IV) 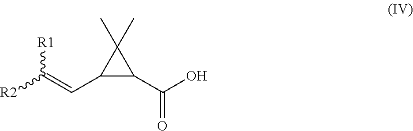
with an alcohol of the formula (V) 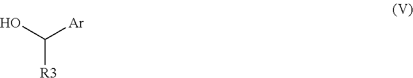
in the presence of a catalyst selected from zirconium containing catalysts, hafnium containing catalysts, iron containing catalysts, cobalt containing catalysts, tin containing catalysts, titanium containing catalysts, ammonium salt catalysts, and boronic acid containing catalysts.

2. The process according to claim 1 wherein the catalyst is a zirconium compound, a hafnium compound, an ammonium salt or a boronic acid compound.

3. The process according to claim 2 wherein the catalyst is a zirconium (IV) compound.

4. The process according to claim 3 wherein the catalyst is ZrCl₄ or a solvent complex thereof.

5. The process according to claim 1, wherein the catalyst is ZrCl₄ or a solvent complex thereof, HfCl₄ or a solvent complex thereof, Ph₂NH₂⁺OTf⁻ or boric acid.

6. The process according to claim 1, wherein R1 and R2 are independently selected from hydrogen, halogen, C1-C4 alkyl and C1-C4 haloalkyl.

7. The process according to claim 1, wherein R3 is selected from hydrogen, cyano or C3-C4 alkynyl.

8. The process according to claim 1, wherein Ar is phenyl substituted with one or more groups selected from hydrogen, halogen, hydroxyl, nitro, cyano, C1-C4 alkyl, C1-C4 haloalkyl, and phenoxy.

9. The process according to claim 1 wherein the carboxylic acid of formula (IV) has the formula (VIII)

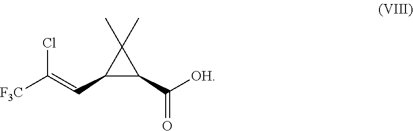

10. The process according to claim 1 wherein the alcohol of formula (V) has the formula (IX)

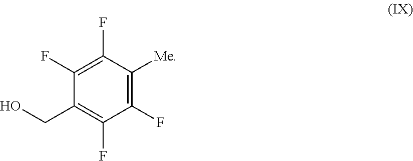

11. The process according to claim 1 wherein the alcohol of formula (V) has the formula (XI)

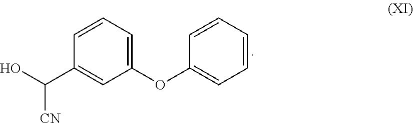

12. The process according to claim 10 for the preparation of tefluthrin (VII)

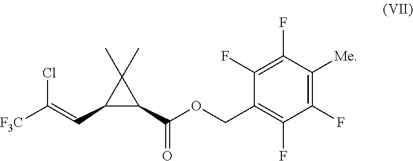

13. The process according to claim 11 for the preparation of lambda cyhalothrin (X)

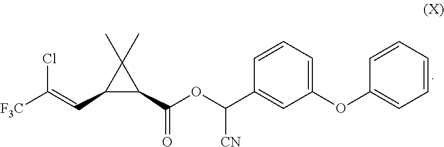

14. The process according to claim 11 for the preparation of gamma cyhalothrin (XIV) [chemical structure]
